SEQUENCE LISTING (9 sequences)

SEQ ID NO: 1 - DNA, 265 bp, Mus Musculus
  misc_feature (0)...(0): Isolate P815 Mouse Mastocytoma cell line, strain DBA, ATCC Deposit No. TIB-64
tacacatcgagtcccggaaatcaaagcaaagaaattcagaatttgagatatttgttgactgcgacatcagccgagaacagttgaatgacatcttccccctgctgaagtcgcacgccaccgtcctctcggtggactcgcccgatcagctcactgcgaaggaagacgttatggagactgtcccttggtttccaaagaagatttctgacctggacttctgcgccaacagagtgctgttgtatggatccgaacttgacgccgaccaccc

SEQ ID NO: 2 - DNA, 265 bp, Rattus Rattus
tacatattgagtcccggaaatcgaagcgaagaaactcagaatttgagatttttgtggactgcgacatcaaccgagaacagctgaatgacatcttccccctgctaaagtcccacaccacggtcctctctgtggactcgcccgatcagctccctgaaaaggaagatgttatggagactgtcccttggttcccaaagaagatttctgacctggacttctgcgccaacagagtgctgttgtacggatccgaactcgacgcggaccactg

SEQ ID NO: 3 - DNA, 265 bp, Homo Sapiens
tacatatcgagtcccgaaaatcaaaaagaagaaactcagaatttgagatttttgttgactgtgacatcaacagagaacaattgaatgatatttttcatctgctgaagtctcataccaatgttctctctgtgaatctaccagataattttactttgaaggaagatggtatggaaactgttccttggtttccaaagaagatttctgacctggaccattgtgccaacagagttctgatgtatggatctgaactagatgcagaccactg

SEQ ID NO: 4 - DNA, 1732 bp, Mus Musculus
  misc_feature (0)...(0): Isolate P815 Mouse Mastocytoma cell, strain DBA ATCC deposit No. TIB-64
gagtgggtgagtgggatagcccctccctgggacatcggatcagaagactcccagcaaggacgggatcaacttctagtaggaaccagattcaccatgattgaagacaacaaggagaacaaagagaacaaagaccattcctccgaaagagggagagtgactctcatcttctccttggagaatgaagtcggaggactcataaaagtgctgaaaatcttccaggagaatcatgtgagcctgttacacatcgagtcccggaaatcaaagcaaagaaattcagaatttgagatatttgttgactgcgacatcagccgagaacagttgaatgacatcttccccctgctgaagtcgcacgccaccgtcctctcggtggactcgcccgatcagctcactgcgaaggaagacgttatggagactgtcccttggtttccaaagaagatttctgacctggacttctgcgccaacagagtgctgttgtatggatccgaacttgacgccgaccaccctggcttcaaagacaatgtctatcgtagaagacgaaagtattttgcagagttggctatgaactacaaacatggggaccccattcccaagattgaattcacggaagaagagattaagacctgggggaccatcttccgagagctaaacaaactctacccgacccacgcctgcagggagtacctcagaaacctccctttgctctcaaaatactgtggctatcgggaagacaacatcccgcaactggaggatgtctccaactttttaaaagaacgcactgggttttccatccgtcctgtggctggttacctctcaccgagagattttctgtcggggttagcctttcgagtctttcactgcactcagtatgtgagacacagttcagatcccctctacactccagagccagacacctgccatgaactcctaggccacgttcctctcttggctgaacccagttttgctcaattctcccaagaaattggcctggcttcccttggagcttcagaggagacagttcaaaaactggcaacgtgctactttttcactgtggagtttgggctgtgcaaacaagatggacagctgagagtctttggggccggcttgctttcttccatcagtgaactcaaacatgcactttctggacatgccaaagtcaagccctttgatcccaagattgcctgtaaacaggaatgtctcatcacgagcttccaggatgtctactttgtatctgagagctttgaagatgcaaaggagaagatgagagaatttgccaagaccgtgaagcgcccgtttggactgaagtacaacccgtacacacagagtgttcaggttctcagagacaccaagagcataactagtgccatgaatgagttgcggtatgaccttgatgtcatcagtgatgccctcgctagggtcaccaggtggcccagtgtgtgatggtttccagtgcatatccaaaaagcctttgagcatcagtctagagccagggctagttcttgcttcccctgaagacctgcttggggagggacagcagctcccagcttagcaatgtctctcgcctctctccatattcaatcactcactctctctgaaaatgcacacctggaactgcttatcttctacttctgttttgtcttctggaacctgctgagggaaatatagttcacgtgccacgtgatgcccaggacacacatttaaaatattttatttcattaaaatgtaattgaatcat

SEQ ID NO: 5 - DNA, 1344 bp, Mus Musculus
  misc_feature (0)...(0): Isolate P815 Mouse Mastocytoma, cell line, Strain DBA, ATCC, Deposit No. TIB-64
atgattgaagacaacaaggagaacaaagagaacaaagaccattcctccgaaagagggagagtgactctcatcttctccttggagaatgaagtcggaggactcataaaagtgctgaaaatcttccaggagaatcatgtgagcctgttacacatcgagtcccggaaatcaaagcaaagaaattcagaatttgagatatttgttgactgcgacatcagccgagaacagttgaatgacatcttccccctgctgaagtcgcacgccaccgtcctctcggtggactcgcccgatcagctcactgcgaaggaagacgttatggagactgtcccttggtttccaaagaagatttctgacctggacttctgcgccaacagagtgctgttgtatggatccgaacttgacgccgaccaccctggcttcaaagacaatgtctatcgtagaagacgaaagtattttgcagagttggctatgaactacaaacatggggaccccattcccaagattgaattcacggaagaagagattaagacctgggggaccatcttccgagagctaaacaaactctacccgacccacgcctgcagggagtacctcagaaacctccctttgctctcaaaatactgtggctatcgggaagacaacatcccgcaactggaggatgtctccaactttttaaaagaacgcactgggttttccatccgtcctgtggctggttacctctcaccgagagattttctgtcggggttagcctttcgagtctttcactgcactcagtatgtgagacacagttcagatcccctctacactccagagccagacacctgccatgaactcctaggccacgttcctctcttggctgaacccagttttgctcaattctcccaagaaattggcctggcttcccttggagcttcagaggagacagttcaaaaactggcaacgtgctactttttcactgtggagtttgggctgtgcaaacaagatggacagctgagagtctttggggccggcttgctttcttccatcagtgaactcaaacatgcactttctggacatgccaaagtcaagccctttgatcccaagattgcctgtaaacaggaatgtctcatcacgagcttccaggatgtctactttgtatctgagagctttgaagatgcaaaggagaagatgagagaatttgccaagaccgtgaagcgcccgtttggactgaagtacaacccgtacacacagagtgttcaggttctcagagacaccaagagcataactagtgccatgaatgagttgcggtatgaccttgatgtcatcagtgatgccctcgctagggtcaccaggtggcccagtgtgtga

SEQ ID NO: 6 - protein, 447 aa, Mus Musculus
MIEDNKENKENKDHSSERGRVTLIFSLENEVGGLIKVLKIFQENHVSLLHIESRKSKQRNSEFEIFVDCDISREQLNDIFPLLKSHATVLSVDSPDQLTAKEDVMETVPWFPKKISDLDFCANRVLLYGSELDADHPGFKDNVYRRRRKYFAELAMNYKHGDPIPKIEFTEEEIKTWGTIFRELNKLYPTHACREYLRNLPLLSKYCGYREDNIPQLEDVSNFLKERTGFSIRPVAGYLSPRDFLSGLAFRVFHCTQYVRHSSDPLYTPEPDTCHELLGHVPLLAEPSFAQFSQEIGLASLGASEETVQKLATCYFFTVEFGLCKQDGQLRVFGAGLLSSISELKHALSGHAKVKPFDPKIACKQECLITSFQDVYFVSESFEDAKEKMREFAKTVKRPFGLKYNPYTQSVQVLRDTKSITSAMNELRYDLDVISDALARVTRWPSV

SEQ ID NO: 7 - DNA, 24 bp, Artificial Sequence
  Primer, MTPH-874F
cacagttcagatcccctctacact

SEQ ID NO: 8 - DNA, 27 bp, Artificial Sequence
  Primer, MTPH-962R
aggagttcatggcaggtgtctggctct

SEQ ID NO: 9 - DNA, 27 bp, Artificial Sequence
  Primer, MTPH-926T
aggagttcatggcaggtgtctggctct

(19) United States
(12) Patent Application Publication
(10) Pub. No.: US 2006/0275761 A1
(43) Pub. Date: Dec. 7, 2006

(54) Tryptophan hydroxylase assay

(76) Inventors: Janet Clark, Matawan, NJ (US); Susan P. Rohrer, Scotch Plains, NJ (US); Stephen E. Alves, New York, NY (US)

Correspondence Address: MERCK AND CO., INC, P O BOX 2000, RAHWAY, NJ, 07065-0907, US

(21) Appl. No.: 10/528,309

(22) PCT Filed: Sep. 15, 2003
(86) PCT No.: PCT/US03/29320

§ 371 Date: Mar. 17, 2005

(30) Foreign Application Priority Data
Sep. 19, 2002 (US) US604120945

(51) Int. Cl. C12Q 1/68

(52) U.S. Cl. 435/6

(57) ABSTRACT

The present invention relates to methods of evaluation and detection of the expression of tryptophan hydroxylase (TPH) gene and induction of TPH by estrogen receptor-beta (ERβ) agonists, utilizing TPH riboprobe and/or primers.

### BACKGROUND OF TH INVENTION

The invention pertains to evaluation and detection of the expression of tryptophan hydroxylase (TPH) gene and induction of TPH by estrogen receptor-beta (ERβ) agonists.

Throughout this disclosure, the term “TPH” refers to TPH isoform one (TPH1).

Serotonin is a key neurotransmitter in the central nervous system, and dysregulation of serotonergic pathways has been implicated in the pathogenesis of many complex psychiatric diseases. Polymorphisms of many of the genes involved in serotonin biosynthesis, catabolism, and response have been reported, suggesting that genetic variability may underlie the development of diseases such as, depression, schizophrenia, obsessive compulsive disorder, and suicide. A number of single-gene polymorphisms in serotonergic pathways have been examined in these and other diseases, but to date results from this candidate gene approach have been disappointing. Although this may be because the detection of a small effect may require the analysis of large numbers of patients and controls, an alternative explanation is that the clinical importance of a single subtle genetic variant may be overlooked unless other functionally related genes are studied in tandem.

Tryptophan hydroxylase (TPH) is the rate-limiting enzyme in serotonin biosynthesis (Grahame-Smith, Biochem. Biophys. Res. Commun. 16:586-592 (1964)). As such, the TPH gene is a likely target for modulation of serotonergic function, which has been associated with several psychiatric disorders (Azmitia, et al., J. Clin. Psychiatry, 52:(12, Suppl.) 4-16 (1991); Hart, et al., Mol. Cell. Neurosci. 2:71-77 (1991); Owens, et al., Clin. Chem. 40:288-295 (1994)). TPH has been the candidate gene of focus in many of the association studies of suicidal behavior. Association between the gene that codes for TPH enzyme in the synthesis of serotonin has been investigated by several investigators. The results continue to point to the substantial role that the gene that codes for TPH play in the serotonin biosynthesis.

The effect of estrogen and progesterone on the protein expression of the rate-limiting enzyme in serotonin synthesis, TPH, and the level of serotonin in the hypothalamic terminal field has been examined in guinea pigs. Investigators also have examined whether serotonin neurons of guinea pigs contain ovarian steroid receptors (estrogen receptor alpha (ERα), estrogen receptor beta (ERβ), progestin receptors) that could directly mediate the actions of estrogen or progesterone. The localization of ERβ, but not ERα or progestin receptor, in the dorsal raphe nucleus suggests that estrogen acting via ERβ within serotonin neurons increases expression of TPH, but that progesterone acting via other neurons and transsynaptic stimulation may effect changes in TPH enzymatic activity, which in turn, is suspected to play a role in serotonin biosynthesis (Lu, et al., Endocrine 11(3):257-67 (1999)).

Determining the level of TPH mRNA transcript will be important in detecting and measuring abnormal serotonergic function, as well in evaluating agonists that can induce TPH message. However, it has been technically difficult to measure TPH mRNA levels in central serotonergic neurons due to its low levels (Darmon, et al., J. Neuroclie 51:312-316 (1988); Jacobs, et al., Physiol. Rey. 72:165-229 (1992)).

### SUMMARY OF THE INVENTION

The present invention provides detection and evaluation of the expression of tryptophan hydroxylase (TPH) gene induced by a variety of molecules having ERβ agonist activity.

In another aspect, the invention provides methods for screening a test molecule for ERβ agonist activity, wherein the method comprises the steps of contacting the test molecule and a TPH mRNA riboprobe with a biological sample; determining the level of transcription of TPH in the sample by hybridization, thereby generating data for a test level; and comparing the test level to a control level, wherein an increase in TPH transcript level in the sample relative to the control indicates ERG agonist activity of the test molecule and/or an induction of TPH message.

Yet in another aspect, the invention provides methods for screening a test molecule for ERβ agonist activity, wherein the transcription of TPH is induced by an ERβ agonist.

Yet in another aspect, the invention provides methods for screening a test molecule for ERβ agonist activity comprising the steps of contacting the test molecule and TPH primers with a biological sample; determining the level of transcription of TPH in the sample by RT-PCR, thereby generating data for a test level; and comparing the test level to a control level, wherein an increase in TPH transcript level in the sample relative to the control indicates ERβ agonist activity of the test molecule and/or an induction of TPH message.

Still in another aspect, the invention provides methods for screening a test molecule for ERβ agonist activity, wherein the expression of TPH is induced by an ERβ agonist.

In one aspect of the invention, there are provided kits for TPH assay that comprise a TPH mRNA riboprobe. The kit, according to the invention, can further comprise dNTPs, a hybridization buffer, and/or commonly used hybridization reagents. In a further aspect of the invention, the control level is obtained by measuring the level of TPH mRNA transcripts in a sample that has been vehicle treated.

In TPH assay kit, as described herein, comprises a TPH mRNA riboprobe and can include a hybridization buffer, wherein in situ hybridization histochemistry is employed for the assay, wherein the concentration of the riboprobe is between about 1 and about 500 ng/ml, wherein the concentration of the riboprobe can be between about 20 and about 200 ng/ml, and wherein the concentration of the riboprobe can be also between about 50 and about 100 ng/ml.

According to another aspect, the invention provides kits for detection and/or assay of transcription of TPH gene in a biological sample, in vivo or in vitro, that comprise TPH riboprobe, and optionally one or more of dNTPs, a hybridization buffer, and hybridization reagents.

Yet, in another aspect of the invention, there are provided kits for TPH assay that comprise TPH primers. The TPH assay kit, as described herein, can comprise one or more of additional components selected from the group consisting of a reverse transcriptase buffer, a reverse transcriptase enzyme, a PCR-buffer, dNTPS, a thermostable DNA polymerase, and commonly used PCR reagents.

The kit, according to the invention, is used to amplify target nucleic acids, wherein RT-PCR is employed, wherein the target nucleic acid is RNA, wherein the concentration of the primer is between about 1 and about 500 ng/ml. In a further aspect of the invention, the RT-PCR employed is real time RT-PCR.

Yet, according to another aspect, the invention provides kits for detection and/or assay of transcription of TPH gene in a biological sample, in vivo or in vitro, that comprise TPH primers, and optionally one or more of reverse transcriptase buffer, reverse transcriptase, PCR-buffer, dNTPS, a thermostable DNA polymerase, and commonly used PCR reagents, wherein the concentration of the TPH primers are between about 1 and about 500 ng/ml.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while often indicating preferred aspects of the invention, are given by way of illustration only, since various changes and modifications within the spirit and scope of the invention will become apparent to those skilled in the art from this detailed description.

### DETAILED DESCRIPTION OF THE INVENTION

The present invention provides methods of detecting TPH mRNA transcripts using a labeled TPH-riboprobe, which comprises a fragment of TPH coding sequence. The riboprobe, which is able to detect the TPH message, is annealed to RNA in a tissue sample, for example from rodent, and subsequently digested with the enzyme RNase A. The riboprobe need not be the full length of the TPH mRNA or TPH gene, but rather can be a fragment.

The term “probe” refers to a defined nucleotide sequence, such as DNA, RNA, PNA, or other derivative molecules, of any length, which binds through complementary base pairing to a subsequence of a target nucleic acid. A probe that is designed based on amino acid and DNA sequence and transcribed from that DNA sequence is referred to as a “ribourobe”. As used herein, a probe may include natural (i.e., A, G, C, or T) or modified bases (7-deazaguanosine, inosine, etc.). In addition, the bases in a probe may be joined by a linkage other than a phosphodiester bond, so long as it does not unduly interfere with hybridization. It will be understood by one of skill in the art that probes will typically substantially bind target sequences lacking complete complementarity with the probe sequence depending upon the stringency of the hybridization conditions. The probes are preferably directly labelled as with isotopes or indirectly labelled with labels such as biotin, to which a streptavidin complex may later bind. By assaying for the presence or absence of the probe, one can detect the presence or absence of the target. The probe can be labelled by any standard technique known in the art, such as radiolabelling, fluorescence, and enzyme linked immunoassays using labels known in the art, such as radioisotopes, FITC or other fluorochrome markers, enzymes, biotin, digoxigenin, or other molecules capable of detection.

The term “TPH-riboprobe”, as described herein, includes any riboprobe made based on TPH mRNA sequence, TPH gene sequence, or any fragment thereof, including, TPH nucleic acid (DNA and RNA), their polymorphic variants, alleles, mutants, and interspecies homologs that have (i) substantial nucleotide sequence homology with the nucleotide sequence of the GenBank Accession No. J04758 (TPH mRNA, complete cds, SEQ ID NO:4); or (ii) at least 65% sequence homology with the amino acid sequence of the GenBank protein_id AAA63401.1 (SEQ ID NO:6); or (iii) substantial nucleotide sequence homology with the nucleotide sequence as set forth in SEQ ID NO:5; or (iv) substantial sequence homology with the encoded amino acid sequence (for example, SEQ ID NO:6). For example, a 265 base TPH mRNA riboprobe (see SEQ ID NO: 1) was used to perform in situ hybridization on cryostat-cut 16 μm thick coronal DRN sections. The clone is 90% homologous to the corresponding region of the rat TPH cDNA (Darmon, et al., J. Neurochem. 51:312-316 (1988); see SEQ ID NO:2) and 81% to the human sequence (Ledley, et al., Cell Mol. Genetics 13:575-580 (1987); see SEQ ID NO:3):

| | | |
|---|---|---|
| mTPH | TACACATCGA GTCCCGGAAA TCAAAGCAAA GAAATTCAGA ATTTGAGATA | (SEQ ID NO:1) |
| rTPH | ----T--T-- ---------- --G----G-- ----C----- ---------T | (SEQ ID NO:2) |
| hTPH | ----T----- ------A--- -----AGG-- ----C----- ---------T | (SEQ ID NO:3) |
| mTPH | TTTGTTGACT GCGACATCAG CCGAGAACAG TTGAATGACA TCTTCCCCCT | |
| rTPH | -----G---- ---------A ---------- C--------- ----T----- | |
| hTPH | ---------- -T-------A -A-------A --------T- -T--T-AT- | |
| mTPH | CCTGAAGTCG CACGCCACCG TCCTCTCGGT GGACTCGCCC GATCAGCTCA | |
| rTPH | ---A-----C ---A----G- -------T-- ---------- ---------C | |
| hTPH | ---------T --TA---AT- -T-----T-- -A-TCTA--A ---A-TT-T- | |
| mTPH | CTGCGAAGGA AGACGTTATG GAGACTGTCC CTTGGTTTCC AAAGAAGATT | |
| rTPN | ---AA----- ---T------ ---------- -------C-- ---------- | |
| hTPN | --TT------ ---T-G---- --A-----T- ---------- ---------- | |
| mNPH | TCTGACCTGG ACTTCTGCGC CAACAGAGTG CTGTTGTATG GATCCGAACT | |
| rTPH | ---------- ---------- ---------- --------C- ---------- | |
| hTPH | ---------- --CAT--T-- ---------T ---A------ ----T----- | |
| mTPH | TGACGCCGAC CACCC | |
| rTPN | C-----G--- ---TG | |
| hTPH | A--T--A--- ---TG | |

The TPH cDNA sequence (see SEQ ID NO:4) chosen was at the 5′ end of the gene, extending from nucleotide 239 to 503 (GenBank accession no. J04758; SEQ ID NO:5; Stoll, et al., Genomics, 7(1):88-96 (1990).

The antisense and sense probes were synthesized using ³⁵S-labeled UTP incorporated into cRNA. The probe was transcribed using a cDNA template containing RNA polymerase sequence extensions for T7 (antisense) and T3 (sense). The template was amplified from mouse brain cDNA using primers against the mouse sequence.

The term “primer” refers to an oligonucleotide, whether natural or synthetic, capable of acting as a point of initiation of DNA synthesis under conditions in which synthesis of a primer extension product complementary to a nucleic acid strand is initiated, i.e., in the presence of four different nucleotide triphosphates and an agent for polymerization (i.e., DNA polymerase or reverse transcriptase) in an appropriate buffer and at a suitable temperature. A primer is preferably an oligodeoxyribonucleotide and is single stranded for maximum efficiency in amplification, but may also be double stranded. If double stranded, the primer is first treated to separate its strands before being used to prepare extension products. The exact length of a primer will depend on many factors, but typically ranges from 15 to 25 nucleotides. Short primer molecules generally require cooler temperatures to form sufficiently stable hybrid complexes with the template. A primer need not reflect the exact sequence of the template, but must be sufficiently complementary to hybridize with a template. An example of a non-complementary sequence which may be incorporated into the primer is a sequence which encodes a restriction enzyme recognition site (see U.S. Pat. No. 4,800,159).

The term “TPH-Primers”, as described herein, includes any primers made based on TPH mRNA sequence, TPH gene sequence, or any fragment thereof, including, TPH nucleic acid (DNA and RNA) including complementary forward or reversed sequences, their polymorphic variants, alleles, mutants, and interspecies homologs that have (i) substantial nucleotide sequence homology with the nucleotide sequence of the GenBank Accession No. J04758 (TPH mRNA, complete cds, SEQ ID NO:4); or (ii) at least 65% sequence homology with the amino acid sequence of the GenBank protein_id AAA63401.1 (SEQ ID NO:6); or (iii) substantial nucleotide sequence homology with the nucleotide sequence as set forth in SEQ ID NO:5; or (iv) substantial sequence homology with the encoded amino acid sequence (for example, SEQ ID NO:6). For example, primers can be made based on SEQ ID NO:7 and SEQ ID NO:8.

A primer can be labeled, if desired, by incorporating a label detectable by spectroscopic, photochemical, biochemical, immunochemical, or chemical means. For example, useful labels include ³²P, ³⁵S, fluorescent dyes, electron-dense reagents, enzymes (as commonly used in ELISAs), biotin, or haptens or proteins for which antisera or monoclonal antibodies are available. A label can also be used to “capture” the primer so as to facilitate the immobilization of either the primer or amplified DNA on a solid support.

The term “nucleic acid” refers to a deoxyribonucleotide or ribonucleotide polymer in either single- or double-stranded form, and unless otherwise limited, would encompass known analogs of natural nucleotides which can function in a similar manner as naturally occurring nucleotides. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

In general, a gene is a region on the genome that is capable of being transcribed to an RNA that either has a regulatory function, a catalytic function, and/or encodes a protein. An eukaryotic gene typically has introns and exons, which may organize to produce different RNA splice variants that encode alternative versions of a mature protein. The skilled artisan will appreciate that the present invention encompasses all TPH-encoding transcripts that may be found, including splice variants, allelic variants and transcripts that occur because of alternative promoter sites or alternative poly-adenylation sites. A full-length gene or RNA therefore encompasses any naturally occurring splice variants, allelic variants, other alternative transcripts, splice variants generated by recombinant technologies which bear the same function as the naturally occurring variants, and the resulting RNA molecules. An allele is one of several alternate forms of a gene, which occupies a particular locus on a chromosome.

The term “fragment”, including TPH gene fragment, can be any portion from the gene, which may or may not represent a functional domain, for example, a catalytic domain, a DNA binding domain, etc. A fragment may preferably include nucleotide sequences that encode for at least 25 contiguous amino acids, and preferably at least about 30, 40, 50, 60, 65, 70, 75 or more contiguous amino acids or any integer thereabout or therebetween.

The term “isolated” refers to material that is substantially free from components which normally accompany it as found in its native state. For instance, an isolated DNA molecule is a fragment of DNA that has been separated from the chromosomal or genomic DNA of an organism. Isolation also is defined to connote a degree of separation from original source or surroundings. For example, a cloned DNA molecule encoding an avidin gene is an isolated DNA molecule. Another example of an isolated DNA molecule is a chemically-synthesized DNA molecule, or enzymatically-produced cDNA, that is not integrated in the genomic DNA of an organism. Isolated DNA molecules can be subjected to procedures known in the art to remove contaminants such that the DNA molecule is considered purified, that is towards a more homogeneous state.

The term “purified” refers to material that is free to varying degrees from components which normally accompany it as found in its native state. “purify” denotes a degree of separation that is higher than isolation. A “purified” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized Purity and homogeneity are typically determined using analytical chemistry techniques, for example, polyacrylamide gel electrophoresis or high performance liquid chromatography. The term purified can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. Various levels of purity may be applied as needed according to this invention in the different methodologies set forth herein; the customary purity standards known in the art may be used if no standard is otherwise specified.

The term “complementary DNA” (cDNA), as used herein, often referred to as a “copy DNA”, is a single-stranded DNA molecule that is formed from an mRNA template by the enzyme reverse transcriptase. Typically, a primer complementary to portions of the mRNA is employed for the initiation of reverse transcription. Those skilled in the art also use the term “cDNA” to refer to a double-stranded DNA molecule that comprises such a single-stranded DNA molecule and its complementary DNA strand.

The term “amplifying” or “amplification” refers to describe both linear and exponential increases in the numbers of a select target sequence of nucleic acid, or a gene, in vivo or in vitro. Amplification can be carried out according to a number of methods well known to those of skill in the art. Examples of such methods include polymerase chain reaction (PCR), ligase chain reaction (LCR), RNA transcription-based amplification systems, reverse transcriptase-PCR (RT-PCR), and the like.

Sample methodologies for employing “Real-time RT-PCR” as described herein is as follows: RNA isolated from a vehicle or compound treated animal tissue or cell is reverse transcribed to generate a cDNA template. The cDNA template is put into a reaction with forward and reverse primers and a fluorescence resonant energy transfer RET) probe with sequence unique to the gene of interest, in this case TPH. During polymerase chain reaction (PCR) the level of fluorescence generated by the probe directly correlates with the level of the RNA of interest in the test sample. Comparison of the level of fluorescence generated for a test sample to that generated by a vehicle sample indicates the relative level of expression of the RNA of interest in the original sample.

The term “recombinant”, when referring to a nucleic acid probe refers to an oligonucleotide which is free of native proteins and nucleic acid typically associated with probes isolated from the cell which naturally contains the probe sequence as a part of its native genome. Recombinant probes include those made by amplification means such as PCR and genetic cloning methods where bacteria are transformed with the recombinant probe.

The term “TPH” refers to TPH nucleic acid (DNA and RNA), protein (or polypeptide), and can include their polymorphic variants, alleles, mutants, and interspecies homologs that have (i) substantial nucleotide sequence homology with the nucleotide sequence of the GenBank Accession No. J04758 (TPH mRNA, complete cds, SEQ ID NO:4); or (ii) at least 65% sequence homology with the amino acid sequence of the GenBank protein_id AAA63401.1 (SEQ ID NO:6); or (iii) substantial nucleotide sequence homology with the nucleotide sequence as set forth in SEQ ID NO:5; or (iv) substantial sequence homology with the encoded amino acid sequence (for example, SEQ ID NO:6). As used in this disclosure, “TPH” refers to TPH isoform 1 (TPH1). TPH isoform 2 (TPH2), as described in Walther, et al., “Synthesis of Serotonin by a Second Tryptophan Hydroxylase Isoform,” Science, Vol. 299 p. 76, is not the subject of this disclosure.

TPH polynucleotides or polypeptides are typically from a mammal including, but not limited to, human, rat, mouse, hamster, cow, pig, horse, sheep, or any mammal. A “TPH polynucleotide” and a “TPH polypeptide,” may be either naturally occurring, recombinant, or synthetic (for example, via chemical synthesis).

TPH polynucleotide or polypeptide sequences are typically from a mammal including, but not limited to, human, rat, mouse, hamster, cow, pig, horse, sheep, or any mammal. A “TPH polynucleotide” and a “TPH polypeptide,” may be either naturally occurring, recombinant, or synthetic (for example, via chemical synthesis).

A “biological subject”, as used herein, is a target biological object obtained, reached, or collected in vivo or in situ, that contains or is suspected of containing nucleic acids or polypeptides of TPH. A biological subject is typically of eukaryotic nature, for example, insects, protozoa, birds, fish, reptiles, and preferably a mammal, for example, rat, mouse, cow, dog, guinea pig, rabbit or chimpanzees.

A “biolopical sample”, as used herein, is a sample obtained from a biological subject, including sample of biological tissue or fluid origin, obtained, reached, or collected in vivo or in situ, that contains or is suspected of containing nucleic acids of TPH. Such samples include, but are not limited to, organs, tissues, fractions and cells isolated from mammals including, mice, and rats. Biological samples may also include sections of the biological sample including tissues, for example, frozen sections taken for histologic purposes. A biological sample is typically of an eukaryotic origin, for example, insects, protozoa, birds, fish, reptiles, and preferably a mammal, for example, rat, mouse, cow, dog, guinea pig, or rabbit, and most preferably a primate, for example, chimpanzees or humans.

The term “control sample”, as used herein, refers to a sample of biological material representative of vehicle-treated animals. The level of TPH in a control sample, is desirably typical of the general population of normal animals of the same species. This sample either can be collected from an animal for the purpose of being used in the methods described in the present invention or it can be any biological material representative of normal animals obtained for other reasons but nonetheless suitable for use in the methods of this invention. In some situations, the control is implicit in the particular measurement. An example of an implicit control is where a detection method can only detect TPH, or the corresponding TPH transcripts, when a level higher than that typical of a normal animal is present. Another example is in the context of an immunohistochemical assay where the control level for the assay is known. Other instances of such controls are within the knowledge of the skilled person.

The term “expression” refers to the biosynthesis of a gene product. For example, in the case of TPH gene, expression involves transcription of the TPH gene into mRNA and the translation of mRNA into one or more polypeptides. Expression of a TPH gene can be indicated by an “increased” or “elevated” level of a TPH polynucleotide or protein compared to a control level of TPH polynucleotide or polypeptide. Comparison may be carried out by statistical analyses on numeric measurements of the expression or it may be done through visual examination of experimental results by qualified researchers.

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res, 19:081, 1991; Ohtsuka et al., J. Biol. Chem, 260:2600-2608, 1985; Rossolini et al., Mol. Cell Probes, 8:91-98, 1994). The term nucleic acid can be used interchangeably with gene, cDNA, mRNA, oligonucleotide, and polynucleotide.

The terms “protein”, “peptide” and “polypeptide” are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides can be chemically synthesized or synthesized by recombinant DNA methods; or, they can be purified from tissues in which they are naturally expressed, according to standard biochemical methods of purification.

A “label” or a “detectable moiety” is a composition that when linked with the nucleic acid or protein molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. A labeled nucleic acid or oligonucleotide probe is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

The term “hybridizing” refers the binding of two single stranded nucleic acids via complementary base pairing under stringent hybridization conditions when that sequence is present in a complex mixture (for example, total cellular or library DNA or RNA). Stringent hybridization conditions refers to conditions under which a probe will hybridize to its target complementary sequence, typically in a complex mixture of nucleic acids, but to no other sequences. Stringent conditions are sequence-dependent and circumstance-dependent; for example, longer sequences can hybridize with specificity at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

Exemplary stringent hybridization conditions can be as following, for example: 4% formaldehyde in 1× PBS buffer, rinsed in 1× PBS, acetylated with 0.2% acetic anhydride in 0.1M triethanolamine, and rinsed in 2× saline sodium citrate (SSC) (2× SSC: 0.3M NaCl, 0.03M sodium citrate). After rinses and ethanol dehydration, sections are hybridized overnight at 55° C. with 5×10⁴ c.p.m. probe/μl. The following morning, sections are washed in 2× SSC, ribonuclease A (RNase A) at 37° C. for 30 min, 2× SSC, and 0.1× SSC at 65° C. Sections are dehydrated using ethanol, and apposed to β-sensitive film for 6 days at room temperature.

The terms “about” or “approximately” in the context of numerical values and ranges refers to values or ranges that approximate or are close to the recited values or ranges such that the invention can perform as intended, such as having a desired amount of TPH riboprobe or TPH primers in a reaction mixture, as is apparent to the skilled person from the teachings contained herein. This is due, at least in part, to the varying properties of nucleic acid compositions, species, age, race, gender, anatomical and physiological variations and the inexactitude of complex biological systems. Thus these terms encompass values beyond those resulting from systematic error.

The term “test molecule” as used herein refers to any compound, composition, nucleic acid, polypeptide, protein, carbohydrate, lipid, lipoprotein, lipopolysaccharide, small molecule, or ERβ agonist, or any combination thereof, that is to be screened for activity on TPH levels.

The ERβ selective agonists of use in the present invention include any ERβ selective agonist known in the art. Non-limiting examples of ERβ selective agonists include compounds described in International Publication WO 01/82923, which is hereby incorporated by reference. According to the invention, it is preferred that the test molecule is active upon the central nervous system (CNS), such as the brain, following systemic administration, i.e. capable of readily penetrating the CNS. Accordingly, a preferred ERβ agonist, according to the present invention, is a CNS-penetrating molecule, compound, composition, nucleic acid, polypeptide, protein, carbohydrate, lipid, lipoprotein, lipopolysaccharide, small molecule, or known ERβ selective agonist, or any combination thereof.

According to one aspect of the invention, the test molecule, as described herein, can be combined with another component, for example, at least one other active ingredient including norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists and atypical anti-depressants, to test, screen, or evaluate the effect of the additional component or the combined composition on TPH activity. In another aspect of the invention, it is preferred that the test molecule or the combined composition as described herein, is active upon the central nervous system (CNS), such as the brain, following systemic administration, i.e. capable of readily penetrating the CNS. Accordingly, a preferred ERβ agonist, according to the present invention, is a CNS-penetrating molecule, compound, composition or known ERβ selective agonist.

TPH Detection and/or Assay Kits:

Reagents employed in the methods of the invention can be packaged into diagnostic kits. Diagnostic kits include labeled/unlabeled probe and/or primers in separate containers. If the probe and/or primer(s) is (are) unlabeled, the specific labeling reagents may also be included in the kit. The kit may also contain other suitably packaged reagents and materials needed for sample preparation and amplification, for example, extraction solutions for the target nucleic acid, buffers, dNTPs, and/or polymerizing means, and reagents for detection analysis, for example, enzymes and solid phase extractants, as well as instructions for conducting the assay. The kit may also include various reaction mixtures useful for the processes disclosed for detecting a target nucleic acid, such as TPH mRNA, in a biological sample, as described herein, including body fluids of human or animal origin, or extracts of any body component of interest.

A kit, for example, for detection and/or assay of transcription of TPH gene, according to the invention, comprise a TPH mRNA riboprobe and reaction buffers. The concentration of the TPH-riboprobe is between about 1 and about 500 ng/ml, as set forth herein, see Example I.

The kit for detection and/or assay of transcription of TPH gene also may comprise TPH primers; reverse transcriptase buffer; reverse transcriptase; PCR-buffer; and a thermostable DNA polymerase, wherein the concentration TPH primers are between about 1 and about 500 ng/ml, as set forth herein, see Example II.

High Throughput Screening of ERβ Agonists.

Conventionally, often chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (UTS) methods are replacing conventional lead compound identification methods.

In one aspect, high throughput screening methods involve providing a library containing a large number of potential ERD agonists (candidate compounds). Such “combinatorial chemical libraries” are then screened in one or more assays, as described herein, to identify those library members particular chemical species or subclasses) that display a desired characteristic activity. The compounds thus identified can serve as conventional “lead compounds” or can themselves be used as potential ERβ agonists.

Devices for the preparation of combinatorial libraries are commercially available (see, e.g., 357 MPS, 390 MPS, Advanced Chem Tech, Louisville, Kent., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Any of the assays for TPH message described herein are amenable to high throughput screening, including TPH-riboprobe based hybridization and TPH-primer based RT-PCR methods. As described above, the ERβ agonists are preferably screened by the methods disclosed herein.

High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configuarable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

The invention is further described by the following examples, which do not limit the invention in any manner.

### EXAMPLE I

### Murine Tryptophan Hydroxylase mRNA Expression: Detection by In situ Hybridization

Animals and Treatment Groups. Female mice (13 to 16 wks of age) are ovariectomized (C57BL/6s from Charles River; ER Knockout animals from Taconic). Animals are fed a soy-free rodent chow, where they are given an additional time to adjust to the new environment. Mice are orally dosed in the morning (once daily for 4 days) with 0.2 cc of vehicle (20% ethanol:30% polyethylene glycol:50% water) or compound (0.1 to 30 mpk for dose response curves; 10 mpk for single-dose experiments); estradiol 17-beta is subcutaneously administered at 0.2 mpk in sesame oil (0.1 cc). Approximately six hours following the fourth dose, mice are deeply anesthetized with ketamine/xylazine, blood is collected via cardiac puncture, allowed to clot, and then serum is collected by centrifugation. The uterus is dissected out of the abdominal cavity, placed on a dissecting board, and fat is removed with a razor blade. The uterus is placed into a Microfuge tube containing 0.9% saline and placed at 4° C. for overnight. The next day, the uteri are removed from the saline, blotted on a napkin and weighed.

Brains for use in the in situ hybridization experiments are removed from the skull and immediately frozen on dry ice.

In situ Hybridization using TPH-riboprobe. A TPH-riboprobe, for example, a 265 base TPH mRNA riboprobe (see for example, SEQ ID NO: 1), was used to perform in situ hybridization on cryostat-cut 16 μm thick coronal dorsal raphe sections. The TPH cDNA sequence chosen was at the 5′ end of the gene, extending from nucleotide 239 to 503 (GenBank accession no. J04758, see SEQ ID NOs.: 4). The antisense and sense probes were synthesized using ³⁵S-labeled UTP (NEN-Dupont, Boston, Mass.) incorporated into cRNA. The probe was transcribed using a cDNA template containing RNA polymerase sequence extensions for T7 (antisense) and T3 (sense). A Nuctrap push column was used for removal of unincorporated nucleotides (Stratagene, La Jolla, Calif.). The template was amplified from mouse brain cDNA (Clontech, Palo Alto, Calif.) using primers against the mouse sequence.

TACACATCGAGTCCCGGAAATCAAAGCAAAGAAATTCAGAATTTGAGATA
TTTGTTGACTGCGACATCAGCCGAGAACAGTTGAATGACATCTTCCCCCT
GCTGAAGTCGCACGCCACCGTCCTCTCGGTGGACTCGCCCGATCAGCTCA
CTGCGAAGGAAGACGTTATGGAGACTGTCCCTTGGTTTCCAAAGAAGATT
TCTGACCTGGACTTCTGCGCCAACAGAGTGCTGTTGTATGGATCCGAACT
TGACGCCGACCACCC-3′.

In situ Hybridization—Protocol and Evaluation. Slides containing 16 μm thick coronal dorsal raphe sections of the mouse brain were briefly post-fixed in 4% formaldehyde in 1×PBS buffer (Ambion, Austin, Tex.), rinsed in 1×PBS, acetylated with 0.2% acetic anhydride in 0.1M triethanolamine, and rinsed in 2× saline sodium citrate (SSC) (2× SSC: 0.3M NaCl, 0.03M sodium citrate). After rinses and ethanol dehydration, sections were hybridized overnight at 55° C. with 5×10⁴ c.p.m. probe/μl. The following morning, sections were washed in 2× SSC, ribonuclease A (RNase A) at 37° C. for 30 min, 2× SSC, and 0.1× SSC at 65° C. Sections were dehydrated using ethanol, and apposed to β-sensitive film (Biomax MR, NEN-Dupont) for 6 days at room temperature.

Autoradiographic images of midbrain sections were anatomically matched between animals, and densitometry was performed using a CCD video camera (Dage-MTI Inc., Mich. City, Ind.) fitted with Nikon lenses (Nikon Canada, Inc.), and the Scion Image Program. The average gray scale optical density (O.D.) reading was obtained by subtracting the background reading outside the region of interest from the O.D. of the dorsal raphe nucleus. Analysis was performed on 3 coronal sections, representing the rostral to caudal extent of the DRN, spanning about 270 μm.

### EXAMPLE II

### Detection and Measurement of Murine Tryptophan Hydroxylase mRNA Expression by Real Time Quantitative PCR Methods

A. Real Time Quantitative PCR Measurement of TPH Message in Murine Dorsal Raphe

Animals and Treatment Groups. Female mice (13 to 16 wks of age) are ovariectomized (C57BL/6s from Charles River; ER Knockout animals from Taconic). Animals are fed a soy-free rodent chow, where they are given an additional time to adjust to the new environment. Mice are orally dosed in the morning (once daily for 4 days) with 0.2 cc of vehicle (20% ethanol:30% polyethylene glycol:50% water) or compound (0.1 to 30 mpk for dose response curves; 10 mpk for single-dose experiments); estradiol 17-beta is subcutaneously administered at 0.2 mpk in sesame oil (0.1 cc). Approximately six hours following the fourth dose, mice are deeply anesthetized with ketamine/xylazine, blood is collected via cardiac puncture, allowed to clot, and then serum is collected by centrifugation. The uterus is dissected out of the abdominal cavity, placed on a dissecting board, and fat is removed with a razor blade. The uterus is placed into a Microfuge tube containing 0.9% saline and placed at 4° C. for overnight. The next day, the uteri are removed from the saline, blotted on a napkin and weighed.

For TPH-Taqman® RNA measurements, the brains are removed from the skull, placed ventral side up in a mouse brain block on ice, and ice-cold razor blades are inserted into the block at 1 mm intervals. The caudal extent of the hypothalamus is used as an anatomical marker for the placement of the first razor blade, and 4 blades are placed in sequential slots, caudally. The four sections are examined and the two that encompass the greatest extent of the dorsal raphe are placed in a Microfuge tube containing RNA-later and placed at 4° C. for overnight.

Murine TPH TaqMan® Primers and Probe Sequences. Murine TFH forward primer is named mTPH-874F, its corresponding sequence is: 5′-CAC AGT TCA GAT CCC CTC TAC ACT-3′ (SEQ ID NO: 7), and it spans nucleotides 874 to 897. The murine TPH reverse primer is named mTPH-962R, its corresponding sequence is: 5′-GCA AAA CTG GOT TCA GCC AA-3′ (SEQ ID NO: 8), and it spans nucleotides 943 to 962. The murine TPH probe is named mTPH-926T, its corresponding sequence is: 5′-AGG AGT TCA TGG CAG GTG TCT GGC TCT-3′ (SEQ ID NO: 9), and it spans nucleotides 900 to 926.

Murine TPH GenBank accession no. J04758 was referenced to design these primers and probe therefore the nucleotide numbering is based on this sequence.

Isolation of total RNA from mouse raphe slices for Taqman® analysis. Samples are stored in RNALater at 4° C. overnight followed by removal of RNALater and storage at −0° C. until isolation of the total RNA (2 slices weigh 25 to 50 mg). Slices are removed from −80° C. and placed in 1.0 ml TRIzol Reagent in FastPrep® processing tubes. Slices are homogenized with one pass at setting 6 for 30 s in FastPrep® 120 homogenizer using green capped tubes with bead matrix followed by 20 s at setting 6 after all samples have been processed. Samples are set at room temperature for 5 min to allow for complete dissociation of nucleoprotein complexes followed by centrifugation of samples at 12,000×g for 5 min at 4° C. Homogenates are transferred to 1.5 ml microfuge tubes and 100 μl BCP (Bromo-3-chloropropane) is added, samples are vortexed for 15 sec. and set at room temperature for 2 to 3 min. Samples are centrifuged at 12,000×g for 15 min at 4° C. The aqueous layer is removed and placed in a new RNAse-free sterile 1.5 ml microfuge tube. A 5 μl of 5 mg/ml glycogen is added to each sample and samples are vortexed. A 500 μl of isopropanol is added to each sample, samples are vortexed for 15 sec., set at room temperature 10 min., followed by centrifugation at 12,000×g for 15 min at 4° C. Supernatants are decanted and pellets washed with 500 μl ice cold 75% ethanol. Samples are centrifuged at 12,000×g for 15 min at 4° C., ethanol decanted and pellets air dried for 10 min. Pellets are resuspended in 30 μl prewarmed RNASecure (60° C.), and samples are heated at 60° C. for 10 min. Samples are stored at −80° C. until DNAse treatment and cDNA synthesis.

B. DNase Treatment and cDNA Preparation with wt Mouse Raphe Slice Total RNA for Taqman® Analysis:

DNase treatment using DNA-free kit (Ambion). A 5 μg of the total RNA sample is aliquotted to each well of a 96 well plate. An 1× DNase I solution is added to each sample (DNase I solution, DNase I buffer, DNase I, H₂O). Reactions are mixed and incubated at 37° C. for 30 min. Reactions are inactivated by addition of DNase Inactivation reagent beads, mixed well at room temperature for 3 min and centrifuged at 2,500 RPM for 1 min at 4° C. (25 μl reactions are run and inactivated with 1/10 volume of inactivation reagent).

Reverse Transcription. A 10 μl of the DNase I-treated total RNA is added to 40 μl of 1× reverse transcription reaction mix (DEPC H₂O, RT buffer, MgCl₂, dNTP mix, random hexamers, RNase inhibitor, and MultiScribe RT) and incubated at 25° C. for 10 min, 48° C. for 30 min, and 95° C. for 5 min. Reverse transcription is halted by the addition of EDTA. Samples are transferred to a storage plate and stored at −20° C.

TaqMan® analysis of raphe slice cDNA for determination of relative levels of murine TPH mRNA. A 2.5 μl of cDNA is added to each well of a 96-well plate with 22.5 μl of TaqMan® reaction mix (1× Universal Master Mix (ABI), 300 nM mTPH-874F and 300 nM mTPH-962R primers, 200 nM mTPH-926T probe, 20 nM forward and reverse rRNA primers, and 100 nM rRNA probe). Samples are run on an ABI PRISM®7700 Sequence Detection Instrument (Applied Biosystems, Foster City, Calif.) and collected data is analyzed using Merck Biometrics TaqManPlus program.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

What is claimed is:

1. A method of screening a test molecule for ERβ agonist activity, wherein the method comprises the steps of:
a) TPH mRNA riboprobe with a biological sample;
b) determining the level of transcription of TPH in the sample by hybridization, thereby generating data for a test level; and
c) comparing the test level to a control level, wherein an increase in TPH transcript level in the sample relative to the control indicates ERβ agonist activity of the test molecule.

2. The method of claim 1, wherein the transcription of TPH is induced by an ERβ agonist.

3. A method of screening a test molecule for ERβ agonist activity comprising the steps of:
a) contacting the molecule and TPH primers with a biological sample;
b) determining the level of transcription of TPH in the sample by reverse transcription and polymerase chain reaction (RT-PCR), thereby generating data for a test level; and
c) comparing the test level to a control level, wherein an increase in TPH transcript level in the sample relative to the control indicates ERβ agonist activity of the test molecule.

4. The methods of claim 3, wherein the expression of TPH is induced by an ERβ agonist.

5. The method according to claim 3, wherein the control level is obtained by measuring the level of TPH mRNA transcripts in a sample that has been vehicle treated.

6. A kit for TPH assay comprises a TPH mRNA riboprobe.

7. The kit of claim 6, further comprises a hybridization buffer.

8. The kit of claim 6, wherein in situ hybridization histochemistry is employed for the assay.

9. The kit of claim 6, wherein the concentration of the riboprobe is between about 1 and about 500 ng/ml.

10. The kit of claim 6, wherein the concentration of the riboprobe is between about 20 and about 200 ng/ml.

11. The kit of claim 6, wherein the concentration of the riboprobe is about 75 ng/ml.

12. A kit for TPH assay comprises TPH primers.

13. The kit of claim 12 further comprises components selected from the group consisting of a reverse transcriptase buffer, a reverse transcriptase enzyme, a PCR buffer, dNTPs, and a thermostable DNA polymerase.

14. The kit of claim 12, wherein RT-PCR is employed for the assay to amplify a target nucleic acid.

15. The kit of claim 14 wherein the RT-PCR is real time RT-PCR.

16. The kit of claim 12, wherein the target nucleic acid is RNA.

17. The kit of claim 12, wherein the concentration of the primer is between about 1 and about 500 ng/ml.

18. The kit of claim 12, wherein the concentration of the primer is between about 20 and about 200 ng/ml.

19. The kit of claim 12, wherein the concentration of the primer is about 75 ng/ml.

20. The kit of claim 12, wherein the concentration of the primer is about 25 ng/ml.
